(19) United States
(12) Patent Application Publication
(10) Pub. No.: US 2006/0051218 A1
(43) Pub. Date: Mar. 9, 2006

(54) Push-pull operated pump for a microfluidic system

(76) Inventor: Herbert Harttig, Neustadt (DE)

Correspondence Address: Roche Diagnostics Corporation, 9115 Hague Road, PO Box 50457, Indianapolis, IN, 46250-0457, US

(21) Appl. No.: 11/220,293

(22) Filed: Sep. 6, 2005

(30) Foreign Application Priority Data
Sep. 6, 2004 (DE) 10 2004 042 987.1

(51) Int. Cl. F04B 43/00; A61M 1/00

(52) U.S. Cl. 417/412; 417/437

(57) ABSTRACT

The present invention generally relates to a micropump for the conveyance of fluidic media at low flow rates. The micropump comprises at least one flow duct. The flow duct also has at least three pinch areas. The micropump also includes at least three actuators for the pumping of fluidic media. The actuators are configured and arranged in such a way that a force can be exerted on the flow duct by at least one of the actuators in each of the three pinch regions. This results in the flow duct being narrowed or completely closed at the pinched points and the fluid which is located in this region is completely or partially displaced from this pinched region.

### REFERENCE TO RELATED APPLICATIONS

The present application claims priority to German Patent Application No. 10 2004 042 987.1, filed Sep. 6, 2004, which is hereby incorporated by reference in its entirety.

### TECHNICAL FIELD

The present invention generally relates to a micropump for the conveyance of fluidic media at low flow rates, in particular in the range of 1 to 1000 nl/min.

### BACKGROUND

Miniaturized pump systems having numerous different operative principles are known from the prior art. A rough distinction can be made between two classes of pumps in accordance with the operative principles.

In a first class, the physico-chemical properties of the fluids are utilized or varied in a controlled way to exert forces on the fluids and to transport the fluids. In particular, phase transitions of the fluids, osmosis or electrical properties are utilized.

For example, DE 100 29 453 C2 describes a pump in which liquid transport takes place as a result of an evaporation of a transport liquid by means of a wettable diaphragm. U.S. 2002/0166592 A1 and U.S. Pat. No. 6,394,759 B1 describe use of electro-osmotic forces for the transport of suitable liquids. Similarly, in U.S. Pat. No. 6,460,974 B1, a liquid consisting of positively and negatively charged molecules is transported by means of alternating electromagnetic fields. In a similar way, in U.S. Pat. No. 6,458,256 B1, describes use of electromagnetic alternating fields to bring about a periodic movement of a phase boundary between two immiscible liquids and thus exert a pumping action on the liquids. By contrast, in U.S. Pat. No. 6,408,884 B1, magnetic properties of specific fluids are utilized in order to move these through a duct by means of magnetic alternating fields.

In addition to pumps that alter the properties of the fluid, it is known from prior art, pumps are know that which are based on local overheating of the media to be transported by means of one or more heating elements. For example, EP 1 363 020 A2 discloses a micropump with heating elements, to evaporated locally the liquid to be transported. An exact control of the flow rate is possible by means of pulsed operation. A similar device is described in U.S. 2003/0021694 A1. Here, too, heating elements locally evaporate part of the liquid to be transported and thus cause pressure changes in the pump system, with the result that the liquid to be transported is propelled forwards.

As mentioned above, in the second class of pumps, external actuators exert pressure fluctuations are induced in a fluidic system in a controlled way by means of one or more and the liquids to be transported are thus sucked in or propelled forwards. This second class can, in turn, be roughly subdivided into pumps which operate according to a peristaltic principle and pumps which have an inflow provided with an inlet valve, a pressure chamber with the actuator or actuators and an outflow provided with an outflow valve.

Various actuator systems can be employed within this second class of pumps. Thus, for example, U.S. Pat. No. 6,395,638 B1 discloses a pump which has a chamber with an inlet valve and with an outlet valve. The volume of the chamber can be increased or reduced in a controlled way by means of an electrostatic actuator, with the result that the pressure of the liquid in the chamber is lowered or raised. Fluid can thus be sucked into the chamber via the inlet valve and ejected again by the outlet valve.

U.S. 2002/0067992 A1 is based on an electrostrictive actuator principle with a peristaltic pumping action. Here, a fluidic duct is used, with a diaphragm consisting of gallium nitride (GaN) and locally deformable as the result of the application of electrical voltage. The cross section of the duct can be varied locally by means of the plurality of electrodes distributed along the duct, so that the liquid in the duct can be propelled forwards by means of a time-variable electrical signal. A similar operative principle is also utilized in U.S. 2002/0081218 A1. Here, the micropump employed is a pump body which has a duct with a wall consisting of viscoelastic material. The wall consisting of viscoelastic material can be deformed locally by means of a plurality of electrodes distributed along the duct, so that a peristaltic wave occurs electrostatically along the longitudinal axis of the duct and the liquid is thus propelled forwards in the duct.

Furthermore, numerous micropumps of this second class which are based on piezoelectric actuators are known from the prior art. Thus, U.S. Pat. No. 6,481,984 B1, U.S. Pat. No. 6,390,791 B1, U.S. Pat. No. 6,416,294 B1 and U.S. 2002/0146330 A1 disclose pump systems of different designs which in each case have a pressure chamber, the volume of which can be varied in a controlled way by means of one or more piezoelectric actuators. U.S. Pat. No. 6,368,078 B2 even discloses a pump system in which three fluidic chambers of this type, the volume of which can be increased or reduced by means of piezoelectric actuators, are employed. The chambers are connected to one another by means of a liquid duct. When the volume of these three chambers is varied periodically with a phase shift, a peristaltic pumping action is achieved and the liquid to be conveyed is thereby propelled forward through the duct.

U.S. 2003/0019833 A1 discloses a pump system in which the cross section of a duct can be varied locally by means of a hydrostatic actuator. The operative principle of these hydrostatic actuators may be based, for example, on the fact that a rise of the pressure in a first duct causes a reduction in the cross section in a second adjacent duct.

Finally, pump systems in which magnetic actuators are used are known from U.S. 2002/0098097 A1 and U.S. 2002/0098122 A1. In this case, a pump chamber which has a flexible diaphragm as a wall is employed. A magnetic element is connected to the diaphragm. Thus, by means of a magnet, the position of the diaphragm can be varied as a result of the attraction or repulsion of the magnetic element, with the result that the volume of the chamber can be influenced in a controlled way.

The prior art described for micropumps has various disadvantages which are manifested particularly in use for diagnostic purposes. Thus, for example, the systems described, which consist of a pumping chamber connected to an inlet and to an outlet valve, have the disadvantage that it is possible for them to operate in only one direction (for example, suction operation). For many applications, however, particularly for organic exchange apparatuses (for example, membrane filters, microdialysers), delivery/suction operation is required.

Furthermore, many of the systems described necessitate the frequent intervention of the user, particularly in the form of a readjustment of the volumetric flow rate. It is precisely for medical diagnostics, however, that maintenance-free systems requiring no user intervention are desirable.

A further disadvantage is that most of the pump systems described have been developed for gases. Where gases are concerned, however, the problem of gas-bubble formation, specific to liquids, does not arise, and therefore pump systems of this type, which are optimized for gases, do not necessarily have optimum functioning for the conveyance of liquids. On the other hand, in turn, many of the pumps for gases have a high dependence of the conveying rate on the temperature and pressure, so that it is sometimes possible only with difficulty to set a constant conveying rate.

Furthermore, most micropumps of the prior art described have a relatively high delivery capacity. Particularly in the range of below 100 nl/min, only a few pump systems are known which can maintain a constant conveying rate over a lengthy period of time. It is precisely in the sector of medical diagnostics, however, that even micropumps with a delivery capacity of 0 to 100 nl/min are required.

Furthermore, the systems described have in each case relatively high construction volumes and offer relatively few approaches for expedient miniaturization. Precisely in the area of portable diagnostic appliances, however, small construction volumes are an essential precondition. Moreover, many of the systems described are relatively complicated and therefore costly. Thus, in particular, systems based on piezoceramics or systems, the production of which requires complicated semiconductor technology, affords scarcely any possibility for cost savings.

It is against the above background that the present invention proves certain unobvious advantages and advancements over the prior art. In particular, the inventor has recognized a need for improvements in Push-Pull operated pump for a microfluidic system.

### SUMMARY

The object of the present invention is, therefore, to avoid the disadvantages of the prior art described and to provide a micropump which is also suitable for liquids and allows delivery/suction operation. Furthermore, the micropump is to have a small construction volume and also be suitable for low conveying rates.

A micropump for the pumping of fluidic media is proposed. The term “micropump” is in this case to be understood as meaning a pump with duct structures, the duct structures having a clear width of no more than one millimetre.

One of the objects of the present invention is a micropump having at least one flow duct with a wall. The flow duct also has at least three pinch areas. The micropump also includes at least three actuators for the pumping of fluidic media. The actuators are configured and arranged in such a way that a force can be exerted on the flow duct by at least one of the actuators in each of the three pinch regions. This results in the flow duct being narrowed or completely closed at the pinched points and the fluid which is located in this region is completely or partially displaced from this pinched region.

In a further advantageous development of the invention, the number of actuators corresponds exactly to the number of pinch regions. The micropump should in this case be configured such that, when a force is exerted by one or more of the actuators in one of the pinch regions, the flow duct narrows, in particular up to a complete closure of the flow duct. A fluid which is located in the flow duct is then displaced completely or partially out of this pinch region. This accordingly also encompasses an expansion of the pinch region by means of suitable actuators, and therefore the term “pinch region” is to be interpreted broadly.

The pinch regions of the micropump are preferably arranged along a longitudinal axis of a flow duct. In this case, the term “longitudinal axis” is not necessarily to be understood as meaning a linear axis, but it may, for example, also be a curved axis. The pinch regions do not in this case necessarily have to be arranged so as to be spaced apart equidistantly, but, in some instances, a non-equidistant spacing is advantageous for the pumping action (in particular, in the case of the magnetic pump, see below). Where a curved axis or a curved flow path is concerned, the term “spacing” or “distance” is in this case to be understood correspondingly as meaning a curved length, for example the curved length of the flow path.

Preferably, the force action of the actuators on the flow duct in pumping operation in the pinch regions takes place periodically at an actuator frequency which is identical for all the actuators. Thus, for example, the actuators may be operated with periodic rectangular activation, in each case a first phase of force exertion being superseded by a second phase, not necessarily having the same length of time, without force exertion or even with negative force exertion. In this case, the force action on the flow duct should take place, in each case with a phase shift, in pinch regions separated spatially from one another, in order to ensure a peristaltic conveyance of the fluid through the flow duct.

It has been shown to be particularly advantageous for the pumping action if the actuators of the micropump operate “in step”. This means that, when a number of N pinch regions (N being an integer, N≧3, see above) is distributed along the longitudinal axis, there occurring between the i'th and the j'th pinch region (with i<j, and i, jε{I, . . . , N}, i and j being integers) a flow path of the length Xᵢ₋ⱼ, the phase angle Φᵢ₋ⱼ of the phase shift of the force action between the i'th and the j'th pinch region is proportional to the length Xᵢ₋ⱼ with a proportionality factor kᵢ₋ⱼ. This proportionality factor kᵢ₋ⱼ should in this case be identical for all i, jε{I, . . . , N}.

It must be pointed out, in this context, that the term “identical” is also to include corresponding tolerances, that is to say the phase shifts may have slight deviations from the said “ideal values”. However, these deviations should not overshoot a value of 20%, preferably even of 10% and, ideally, of 5%.

The said conditions for phase shift ensure, in particular, that, even in the case of a non-equidistant arrangement, for example, actuators “arranged so as to be spaced somewhat further apart” are triggered correspondingly later. Thus, here too, a corresponding peristaltic pumping action can be achieved by the triggering of the actuators “in step”. If, by contrast, all the actuators are in each case distributed equidistantly, that is to say Xᵢ₋ⱼ is identical for all adjacent pinch regions i, j, then the phase shift is identical between all adjacent pinch regions.

A particularly advantageous special case is where, in pumping operation, the at least one flow duct is at any time point closed in at least one of the pinch regions. In this way, a backflow of fluid (leakage) is prevented and continuous pumping operation is maintained. This may be achieved, for example, in that, in each period of the pump, the selected durations of the actuator action, that is to say the selected time during which an actuator exerts a force on the at least one flow duct, is sufficiently long to ensure that, before the last actuator along the flow duct “opens” again, that is to say terminates its force action, the first actuator along the flow duct already “closes” again, that is to say exerts a force on the flow duct again.

In particular, the angular sector of the phase shift may also be “divided equally among the pinch regions”. For example, this may be achieved when the pinch regions are distributed equidistantly along the longitudinal axis. In this case, it is particularly beneficial if the phase angle of the phase shift of the force action between two adjacent pinch actions amounts to 360° divided by the number of pinch regions.

The type of action of the micropump is similar to the type of action of a peristaltic pump. Advantageously, as described above, during operation, at least one of the pinch regions is held at each time point in a pinched-together position. By a pinch region being pinched together, fluid is displaced out of the flow duct in the region of this pinch region and is conveyed into an adjacent pinch region not pinched together. By the actuators being activated correspondingly, fluid can thereby be propelled forwards in the flow duct.

It is particularly advantageous if the at least one flow duct has at least two connection regions, the connection regions being suitable for connecting the at least one flow duct to the supply and discharge system for fluidic media. These connection regions may comprise, for example, screw or plug connectors for the connection of corresponding pipeline systems. Advantageously, however, the micropump itself is integrated completely or partially into a fluidic microstructure, in particular into a fluidic microstructure worked from silicone. In this case, the micropump described, in one of its embodiments, may, for example, be encapsulated with other fluidic microstructure components. In this way, in particular, micropumps with an average volumetric throughput of 0 to 10 000 nl/min, in particular of 0 to 1000 nl/min, and, ideally, of 0 to 100 nl/min, can be produced advantageously and cost-effectively.

A micropump which has at least one drive subassembly and at least one fluidic subassembly has proved particularly advantageous. In this case, the at least one drive subassembly is to comprise completely or partially at least one actuator. Thus, for example, entire actuators or even essential individual parts of these actuators can be accommodated in the drive subassembly. In addition, the drive subassembly may also have further structural elements. The at least one fluidic subassembly is to comprise the at least one flow duct and the at least one wall body. In addition, the fluidic subassembly may also have further elements, in particular parts of the actuators may be arranged in the fluidic subassembly.

The at least one drive subassembly and the at least one fluidic subassembly are to be spatially separable from one another. This separability has the advantage that, for example, the fluidic subassembly can be configured as a cost-effective separable disposable part which is renewed or exchanged for each use. By contrast, the drive subassembly may be reused and then comprises mostly the more costly components of the actuators and, if appropriate, an electronic control.

Furthermore, the fluidic subassembly can also be sterilized separately, this being highly advantageous, above all, in the medical application sector. The entire micropump or else only the fluidic subassembly (especially the flow duct in this case) can then be sterilized selectively. Numerous known sterilization methods may be considered for such sterilization. Thus, for example, γ- or β-rays, steam sterilization (for example, 15 minutes at 121°) or sterilization with ethylene oxide gas may be employed.

Of the various operative principles of actuators which can be used for micropumps of the type described, in particular, the operative principle of the bimetal actuator has proved advantageous. In this case, one or more bimetal actuators can be employed alone or in combination with further actuator types. In particular, it is advantageous if at least one bimetal actuator is employed, the shape of which can be varied as a result of the application of an electrical voltage and/or the conduction of an electrical current. In this case, the magnitude of the force exertion by the bimetal actuator on the at least one flow duct is to be capable of being set by means of the intensity of the electrical voltage and/or of the electrical current at or through the bimetal actuator.

Bimetal actuators which are in the form of tongues have proved particularly suitable. The contacting of the bimetal actuators may take place, for example, via conductor tracks on the casing. When electrical current flows through the bimetal actuator, the latter is deformed and narrows the cross section of the at least one flow duct.

A subdivision of the micropumps which are based on bimetal actuators into a separable drive subassembly and a fluidic subassembly can be achieved, for example, in that the bimetal actuators are accommodated on a separate module separated from the fluidic subassembly having the flow duct. Thus, in particular, the fluidic subassembly can be exchanged or sterilized in a simple way.

Thermal actuators which have at least one heat source have proved to be a further possible and advantageous actuator principle. Advantageously, the thermal actuator comprises the at least one heat source and a homogeneous expansion region of the at least one wall body. In this case, the at least one heat source is to be configured in such a way that it can locally raise the temperature of the homogeneous expansion region of the at least one wall body. As a result, the density of the homogeneous expansion region of the at least one wall body is to be locally varied, in particular reduced, directly, with the result that the at least one expansion region expands and the at least one flow duct is thus narrowed in its cross-sectional area in at least one pinch region.

Advantageously, in this case, the materials used for the at least one wall body in the at least one expansion region are materials with high coefficients of thermal expansion. Particularly advantageous, in this case, are linear coefficients of thermal expansion in the range of 0.01 to 0.09%/K, advantageously 0.02 to 0.04%/K.

In contrast to known thermal actuators, here, action therefore takes place, according to the invention, directly on the wall body of the micropump or on part of the wall body (expansion region). Contrary to this, the prior art (for example, Charlen et al., “Electrothermally Activated Paraffin Microactuators”, Journal of Microelectromechanical Systems, Vol. 11, No. 3, June 2002, pp. 165) discloses paraffin actuators, in which paraffin is used as a substance with a high coefficient of thermal expansion. However, the paraffin has to be encapsulated in a membrane in a complicated way susceptible to faults. The use of a paraffin capsule of this type in a pump system, in particular for medical purposes, would therefore be highly complicated and very risky, since there is always the danger of a bursting of the paraffin capsule. The literature accordingly also does not disclose any use of paraffin capsule actuators of this type for peristaltic pumps.

By contrast, in the thermal actuator according to the invention, which is to be employed specially for pumps, no such capsule is used. There is, here, directly, without a further material first having to be heated and thereby expanded, a local heating of the wall body which correspondingly expands locally and can narrow or close the at least one flow duct. The wall body or part of the wall body (to be precise, the homogeneous expansion region) thus itself becomes part of the thermal actuator.

Various systems can be used as heat sources. Thus, for example, electrical components can be employed, which heat the at least one expansion region of the wall material locally as a result of the action of electromagnetic fields, for example microwave fields or infrared radiation. These heat sources have the advantage, in particular, that they can easily be separated from the rest of the micropump. Thus, for example, a drive subassembly may have one or more radiation sources, whilst a fluidic subassembly comprises the at least one flow duct. Thus, for example, the fluidic subassembly can be exchanged simply and quickly, and the radiation sources on the drive subassembly are not damaged, for example, during steam sterilization.

In particular, however, it is also advantageous to use an electrically heatable resistor as a heat source. In this case, the temperature of the at least one electrically heatable resistor can be set as a result of the application of an electrical voltage and/or the conduction of an electrical current. The magnitude of force exertion on the at least one flow duct by the electrically heatable resistor and therefore the degree of narrowing of the flow duct are then set correspondingly by means of the intensity of the electrical voltage and/or of the electrical current. For the purpose of miniaturization, this at least one electrically heatable resistor may be, in particular, an SMD resistor.

If thermal actuators are employed, it has proved particularly advantageous if at least one thermally insulating, in particular double-walled casing is used. This thermally insulating casing is to surround completely or partially at least one of the pinch regions of a flow duct and also at least one thermal actuator.

The principle of magnetic actuators has proved particularly advantageous as a third actuator principle for use in micropumps of the type described. A micropump is accordingly proposed which has at least one magnetic actuator. Once again, magnetic actuators alone may be used in the micropump, or else magnetic actuators in combination with other operative principles of actuators.

It has proved particularly advantageous if the at least one magnetic actuator has two components: at least one force transmission element and at least one magnetic-field generation element.

The at least one force transmission element should be integrated into the at least one wall body of the micropump and should be configured in such a way that a force can be exerted on the force transmission element as a result of the action of a magnetic field. The force transmission element has the effect, particularly when it is attracted by the magnetic field, of an active return of the wall body of the flow duct after a force action and consequently of a corresponding widening of the flow duct. In particular, the pumping action thereby becomes independent of the elastic return force of the wall body after the termination of pinching together by means of an actuator.

The magnetic-field generation element is to generate a magnetic field of variable intensity at the location of the force transmission element. In particular, this magnetic field is to be configured in such a way that it can act on the at least one force transmission element. In particular, the magnetic-field generation element may have an electromagnet or else a permanent magnet.

In an advantageous embodiment of the invention, the magnetic-field generation element has two components: at least one pumping magnet and at least one control magnet. The at least one pumping magnet is in this case to be configured such that it can be varied in its spatial position and/or its orientation. Thus, it may be, for example, a permanent magnet which can be moved to and fro between two positions by means of a corresponding guide. In this case, the at least one pumping magnet is to generate a magnetic field which acts on the at least one force transmission element. Furthermore, the magnetic field generated by the at least one pumping magnet at the location of the at least one force transmission element is to be dependent on the position and/or orientation of the at least one pumping magnet.

The spatial position and/or the orientation of the at least one pumping magnet are/is to be capable of being set by means of the at least one control magnet. If, for example, the pumping magnet is a permanent magnet which can be moved to and fro between two positions by means of a guide, then, for example, a further permanent magnet may be used as the control magnet. When this control magnet is brought into spatial proximity to the pumping magnet, the pumping magnet, depending on orientation and guidance, is either attracted to the control magnet or repelled from the latter and changes its spatial position correspondingly.

It has proved particularly advantageous if the force transmission element used is an element which consists completely or partially of ferromagnetic material. It may comprise, for example, wafers or discs made from soft iron which are introduced into the at least one wall body of the micropump. If, for example, the pumping magnet used is a permanent magnet, then, when the pumping magnet approaches, the soft-iron wafer is attracted by the latter, with the result that a force, in particular a return force, is exerted on the at least one wall body of the micropump.

Furthermore, for example, particles, in particular a powder, of magnetically soft materials, for example soft-iron powder or carbonyl iron powder, as well as corresponding mixtures of suitable powders or particles, may also be used. Such particles may, in particular, be incorporated locally into the wall body of the micropump, in particular into the silicone. The use of powders has, in particular, the advantage that no sharp macroscopic edges occur (as, for example, in the case of relatively large wafers), at which the surrounding wall body will be damaged.

In particular, it has proved advantageous if the at least one pumping magnet has at least one permanent magnet, advantageously all the permanent magnets having a common magnetic orientation.

It has proved advantageous, in this case, if the at least one pumping magnet can act doubly on the cross section: indirectly magnetically via the force transmission element and additionally directly as a result of mechanical force action. For this purpose, the at least one pumping magnet may be configured such that, in at least one of its positions or orientations, it acts mechanically on the at least one wall body in such a way that the at least one flow duct has, in at least one pinch region, a cross-sectional area other than in at least one other position or orientation of the pumping magnet. Thus, in particular, the pumping magnet may be mounted linearly moveably, in which case, in one position, it compresses, preferably closes, the pinch region and, in another position, relieves the latter.

Furthermore, the control magnet may, in particular, be configured in such a way that it has a spatially moveable device and at least three magnetic elements connected to the spatially moveable device. These magnetic elements should, in particular, have a common preferred axis. Furthermore, the magnetic elements should be divided into groups of identical magnetic polarization, the number of magnetic elements in each group not overshooting the number of permanent magnets reduced by 1.

The spatially moveable device may have, for example, one or more linear displacement units, onto which bar magnets are mounted with their magnetization axis parallel to the displacement axis of the linear displacement units. Thus, the north pole and south pole of the bar magnets can be brought alternately into the vicinity of the pumping magnets, so that these pumping magnets are alternately attracted and repelled.

Alternatively, the spatially moveable device may also have a rotor disc, the magnetic elements being arranged on the rotor disc along a circular path.

Thus, by means of the magnetic actuator or magnetic actuators of the type described, a force is exerted indirectly on the at least one wall body of the micropump: the control magnet or control magnets move or orient the pumping magnet or pumping magnets. As a result of the movement of the pumping magnet or pumping magnets, in turn, the magnetic field is varied at the location of the at least one force transmission element and the force action at the at least one force transmission element (in particular, the attractive force) is thus changed. In this way, as a result of indirect force action, in particular, the cross section of the at least one flow duct can be increased or reduced in a pinch region in the vicinity of this force transmission element.

Furthermore, a micropump system for the pumping of fluidic media is proposed, which has a micropump according to one of the principles described above. Furthermore, the micropump system is to have at least one electronic circuit, by means of which at least one of the actuators can be activated correspondingly, in order thereby to control the cross section of the at least one flow duct in at least one pinch region.

The invention is described below by means of exemplary embodiments which are illustrated in the figures. However, the invention is not restricted to the exemplary embodiments illustrated. The same reference numerals in the figures in this case designate identical components or components corresponding to one another in their function.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 is a block diagram representing the steps involved in manufacturing of the preferred embodiment of the micropump according to the present invention;

FIG. 2 is a side view of a first exemplary embodiment of a micropump for the pumping of fluidic media with three bimetal actuators;

FIG. 3 is a perspective view of a tongue-shaped bimetal actuator of FIG. 2;

FIG. 4 is a sectional illustration of a second exemplary embodiment of a micropump for the pumping of fluidic media with three thermal actuators, perpendicular to the direction of flow of the fluidic media;

FIG. 5 is a side view of the micropump illustrated in FIG. 4;

FIG. 6 is a top view of a third exemplary embodiment of a micropump for the pumping of fluidic media with three magnetic actuators;

FIG. 7 shows a cross-sectional view of the exemplary embodiment illustrated in FIG. 6; and

FIG. 8 shows a longitudinal section of the exemplary embodiment illustrated in FIG. 6.

Skilled artisans appreciate that elements in the figures are illustrated for simplicity and clarity and have not necessarily been drawn to scale. For example, the dimensions of some of the elements in the figure may be exaggerated relative to other elements to help improve understanding of the embodiment(s) of the present invention.

In order that the invention may be more readily understood, reference is made to the following examples, which are intended to illustrate the invention, but not limit the scope therof.

### DETAILED DESCRIPTION

The following description of the preferred embodiment is merely exemplary in nature and is in no way intended to limit the invention or its application or uses.

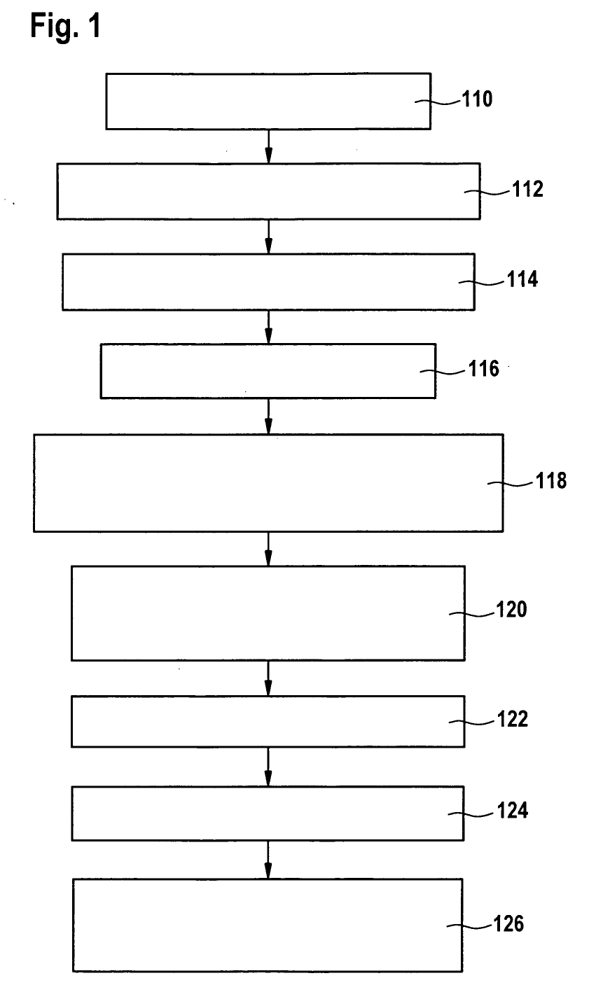

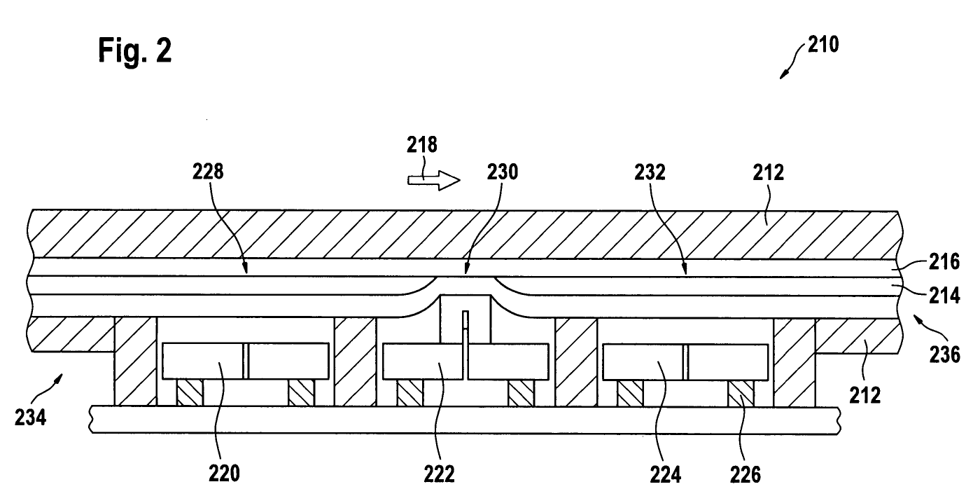

Referring in particular to FIG. 1 a method for the production of a micropump 210 (FIG. 2) for pumping of fluidic media is shown. The method is illustrated by steps that are represented by reference numerals. It must be understood that the method steps described do not necessarily have to be carried out in the sequence illustrated. Furthermore, additional method steps not described may also be carried out. Alternatively, one or more of the method steps may also be carried out simultaneously.

With continued reference to FIG. 1, in order to manufacture the micropump 210, as a first step microinjection-moulding material is used to manufacture at least one casing (step 110). Subsequently, at least one actuator is introduced completely or partially into the casing (step 116). As will be described later, the at least one actuator will help transport the fluidic medium across the micropump.

As shown in step 112 and 114, the at least one surface of the casing and/or of the at least one actuator is coated with an adhesion promoter layer. Thereafter, at least one flow-duct master moulding is introduced completely or partially into the at least one casing (step 120). Alternatively, the flow-duct master moulding may be a wire which is additionally coated with an anti-adhesion layer, for example a PTFE layer (step 118).

Subsequently, the at least one casing is filled completely or partially with an elastic material (step 122). The elastic material may be a combination of a plurality of elastic materials or a combination of an inelastic material with an elastic material. For example, the matrial may be silicone or a cold-curable dimethylsiloxane. If a cold curable material is used, then the method steps comprise an additional step of curing the material (124). After the casing is filled with the elastic material the at least one flow-duct master moulding is removed from the at least one casing again (step 126).

Now referring in particular to FIG. 2, a micropump manufactured in accordance with the steps shown in FIG. 1, is generally represented by refernce numeral 210.

As shown in FIG. 2, the micropump 210, has at least one flow duct 214 with a wall body 216 consisting of silicone. The flow duct 214 is introduced into a casing 212 ((partially of double-walled design to reduce heat losses). As shown, the casing 212 completely or partially surrounding the flow duct 214. As described above and shown in FIG. 1, the casing 212 is produced from a thermoplastic polymer by means of microinjection moulding. Alternatively, the casing may consist of metal, ceramic or of composite materials.

The flow duct 214 of the micropump 210 also comprises at least at least three pinch regions 228, 230 and 232. The cross-sectional area of the flow duct 214 is variable in the pinch regions 228, 230 and 232 by the actions of force on the wall body 216 of the flow duct 214. The flow duct is integrated into a fluidic microstructure, in particular into a microstructure worked from silicone.

The micropump 210 also comprises at least three actuators 220, 222 and 224 for the pumping of fluidic media. The actuators may be a bimetal actuator, a thermal actuator or a magnetic actuator. Alternatively, actuators having different operative principles, may also be employed Although in the figures only three actuators are shown and represented, it must be understood that the micropump 210 may include less or more than three actuators. The micropump 210 may have one flow duct or a plurality of flow ducts, for example flow ducts connected in parallel, which, for example, in each case have their own actuators. The actuators are integrated at least partially into a fluidic microstructure, in particular into a microstructure worked from silicone. This may be, in particular, the same microstructure into which the at least one flow duct is also integrated.

The actuators 220, 222 and 224 are distributed equidistantly in the direction of flow 218 are introduced into the casing 212. As shown in FIG. 2, the actuators 220, 222 and 224 are configured and arranged in such a way that a force can be exerted on the flow duct 214 by at least one of the actuators 220, 222 or 224 in each of the three pinch regions 228, 230 and 232. The actuators are contacted electrically via electric contacts 226 and by means of conductor tracks on the casing (not illustrated). When a current is conducted via the electrical contacts through one of the actuators, the latter straightens up and exerts a force on the wall body 216, so that the flow duct 214 is pinched together at this point and is thus narrowed or completely closed there. FIG. 2 illustrates an actuation of the actuator 222 on the walls 216 so that the flow duct 214 is pinched together at that point 230.

In order to achieve constant volumetric flow rates, it is preferred that the flow duct has a round or elliptical or lenticular cross-sectional area. In such cases, the radius or the lengths of the semi-axes in the cross-sectional area should lie in a range of between 10 μm and 1000 μm. Preferably, in the range of between 10 μm and 500 μm. The cross-sectional area of the flow duct 214 is such that by means of suitable actuators a constant volumetric flow rates of 0-10000 nl/min is achieved. In some cases it is also preferred that a constant volumetric flow rates of 0-1000 nl/min be achieved. In yet some other cases, it is preferred to have a flow constant volumetric flow rate 0-100 nl/min.

The illustrated micropump of the first exemplary embodiment acts essentially in the same way as a line-up of three identical microvalves, since, depending on the activation of the bimetal actuators 220, 222 and 224, the flow duct 214 can be closed in the pinch regions 228, 230 or 232. Each of these microvalves comprises a monolithic body consisting of silicone. The bimetal used in this example is type TB208/110 of Auerhammer Metallwerke GmbH, Auer, Germany. These tongue-shaped bimetal actuators are preferably produced by laser cutting, but other micromechanical methods, such as, for example, etching, sawing or milling, may also be adopted. Electrical contacting takes place via conductor tracks on the casing 212. In the production of the micropump 210, the bimetal actuators 220, 222 and 224 were not provided with an adhesion promoter layer before being sealed in with elastomer, since, in this exemplary embodiment, the bimetal actuators are not embedded into the wall body 216 at all, but are arranged underneath the latter.

In this example, the flow duct 214 has a diameter of approximately 50 to 300 μm, with a round to highly elliptical or lenticular cross section. Depending on the overall size and the frequency of the actuators, the micropump illustrated has a volumetric flow rate of 0 to 10 000 nl/min.

The type of action of this illustrated micropump 210 is based on a peristaltic pumping action. Thus, for example, first, the flow duct 214 can be closed in the pinch region 228. Subsequently, in addition, the flow duct 214 is closed in the pinch region 230, the fluid located in this pinch region 230 being displaced out of the pinch region 230. However, since the fluid cannot escape to the left in the direction of the pinch region 228, it escapes to the right in the direction of the pinch region 232. Thereafter, the flow duct 214 is additionally narrowed in the pinch region 232. Once again, the fluid located there cannot escape to the left in the direction of the pinch region 230, since this pinch region 230 is still closed. It therefore remains only to escape to the right in the direction of flow 218. Overall, during this process, therefore, fluid has been transported from left to right, that is to say in the direction of flow 218. Subsequently, the pinch region 228 is opened again, so that new fluid can penetrate into the pinch region 228 from the left. The process described thereupon recommences.

In the example illustrated here, the electrical contacts 226 of the bimetal actuators are connected to a corresponding electronic control which is not illustrated in FIG. 2. This electrical control, in particular, regulates the time profile of the currents through the bimetal actuators 220, 222 and 224. In this case, the bimetal actuators may be acted upon, for example, with a rectangular periodic voltage. The current profile through the bimetal actuators also correspondingly follows approximately a rectangular profile. A live phase with a first duration is followed by a phase without current through the bimetal actuator, with a second duration, in which case the first and the second duration do not necessarily have to be identical. In the arrangement illustrated, it is expedient to act upon all three bimetal actuators 220, 222 and 224 with the same periodic voltage signal, although a phase shift of 120° occurs between adjacent bimetal actuators. Thus, in particular, the triggering of the bimetal actuator 222 takes place a third period later than the triggering of the bimetal actuator 220. By contrast, the triggering of the bimetal actuator 224, in turn, takes place a third period later than the triggering of the bimetal actuator 222. The peristaltic pumping principle described above can thereby be implemented by means of suitable activation. Other phase shifts can be implemented by means of other activations.

The principle illustrated in FIG. 2 can be extended correspondingly, in that, for example, further bimetal actuators are inserted on the right of the bimetal actuator 224.

A fluidic connection to the micropump illustrated in FIG. 2 may take place in various ways. For this purpose, the micropump 210 has two connection regions 234 and 236. Thus, for example, hoses or capillaries can be connected directly to the flow ducts 214 in the connection regions 234, 236. For this purpose, for example, the wall body 216 of the flow duct 214 may be provided with corresponding threads or connection pieces in the connection regions 234, 236. In this case, there are considerable advantages in producing the wall body 216 from silicone. A flexible reception region for such connections can be integrated without a high outlay. Preferably, however, the micropump 210 is integrated into a fluidic microstructure (not illustrated in FIG. 2) consisting of silicone, in particular is sealed in with the fluidic microstructure and, in a particularly preferred procedure, is produced together with the fluidic microstructure.

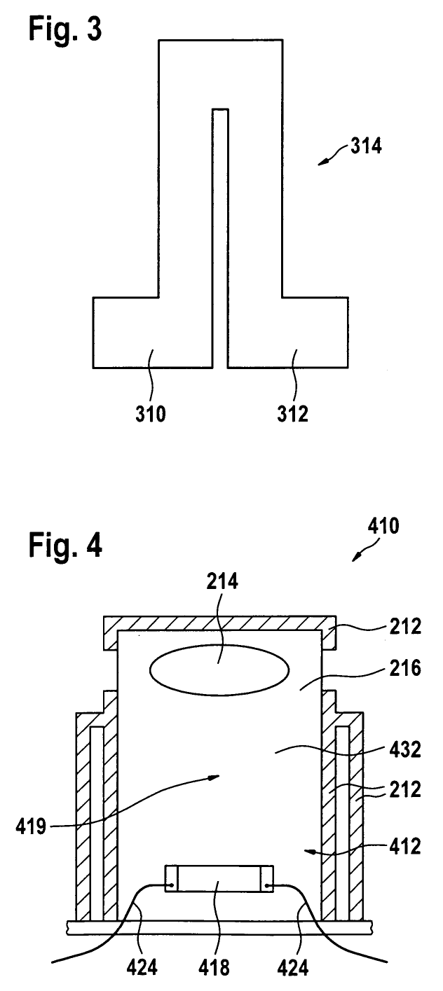

FIG. 3 illustrates a top view of a tongue-shaped bimetal actuator, such as is used in the exemplary embodiment illustrated in FIG. 2. The bimetal actuator has two connection pieces 310 and 312 for electrical contacting and an actuator tongue 314. The connection pieces 310 and 312 are connected to the electrical contacts 226 in FIG. 2. When a current is conducted through the bimetal actuator, the actuator tongue 314 is bent out of the drawing plane in FIG. 3.

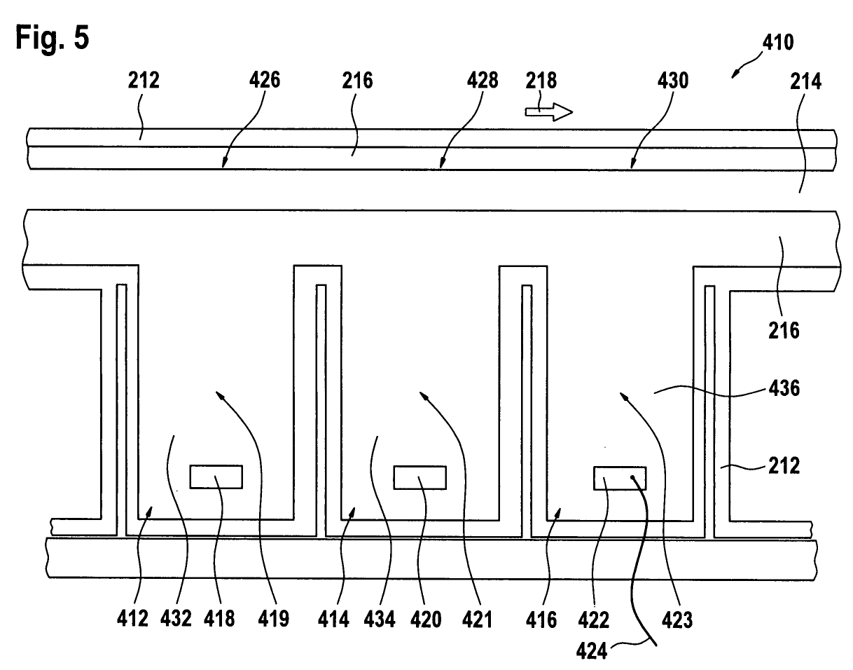

FIGS. 4 and 5 illustrate a second exemplary embodiment of a preferred micropump 410 with thermal actuators 419, 421, 423 for the pumping of fluidic media. Once again, the micropump 410 has a casing 212 produced by microinjection moulding and a flow duct 214 with a wall body 216 consisting of silicone. The micropump is illustrated in FIG. 4 in a sectional illustration perpendicularly to the direction of flow 218 and in FIG. 5 in a sectional illustration parallel to the direction of flow.

Once again, the flow duct 214 has a round to highly elliptical lenticular cross section with a diameter (or a double semi-axis) of between 50 and 300 μm, with the result that (depending on the overall size and actuator control) volumetric flow rates of 0-10 000 nl/min can once again be set. The casing 212 is again designed to be double-walled in part, in order to avoid heat losses. Furthermore, the casing 212 has three cavities 412, 414 and 416 below the flow duct 214. These cavities have in each case a volume of approximately 1 μl and are likewise filled with silicone which forms part of the wall body 216. Elastosil RT 622 of the company Wacker-Chemie GmbH of Burghausen, which has a coefficient of thermal expansion of 0.024%/K, has proved particularly advantageous in this case. The silicone in the cavities 412, 414 and 416 and, directly above these cavities, at the edge of the flow duct 214 thus forms three expansion regions 432, 434 and 436.

An SMD resistor 418, 420 and 422 is embedded in each case into the cavities 412, 414 and 416, the resistors 418, 420 and 422 being contacted electrically via electrical feed lines 424.

When an electrical current flows through one of the resistors 418, 420 or 422, the latter and consequently also the surrounding silicone are heated in the respective expansion regions 432, 434 or 436. Since silicones have a very high coefficient of thermal expansion, the expansion regions 432, 434, 436 expand correspondingly. Owing to this expansion, the flow duct 214 is narrowed or even completely closed in the pinch region 426, 428 or 430 corresponding to the respective expansion region 432, 434, 436.

The pumping action of the second exemplary embodiment illustrated in FIGS. 4 and 5 is once more based on the peristaltic pumping principle in a similar way to the first exemplary embodiment illustrated in FIG. 2. The activation of the thermal actuators 419, 421 and 423 may take place according to the same cyclic principle as in the first exemplary embodiment.

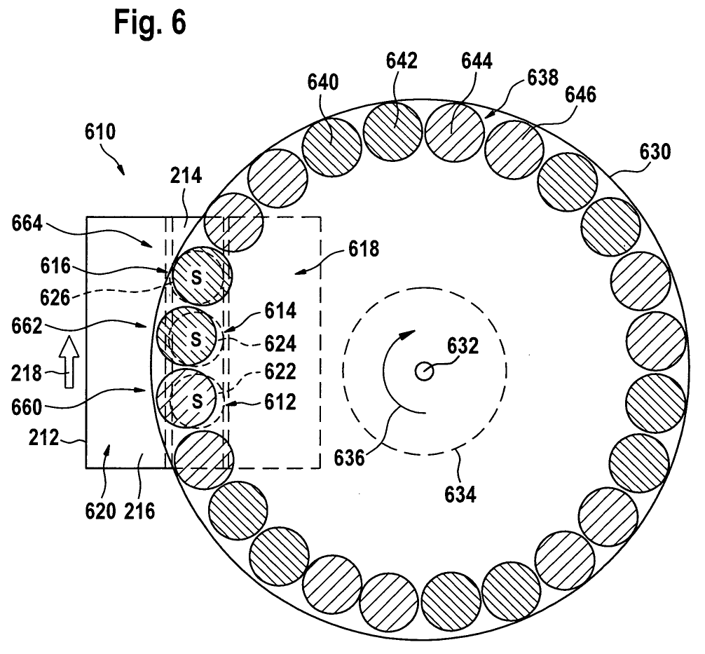

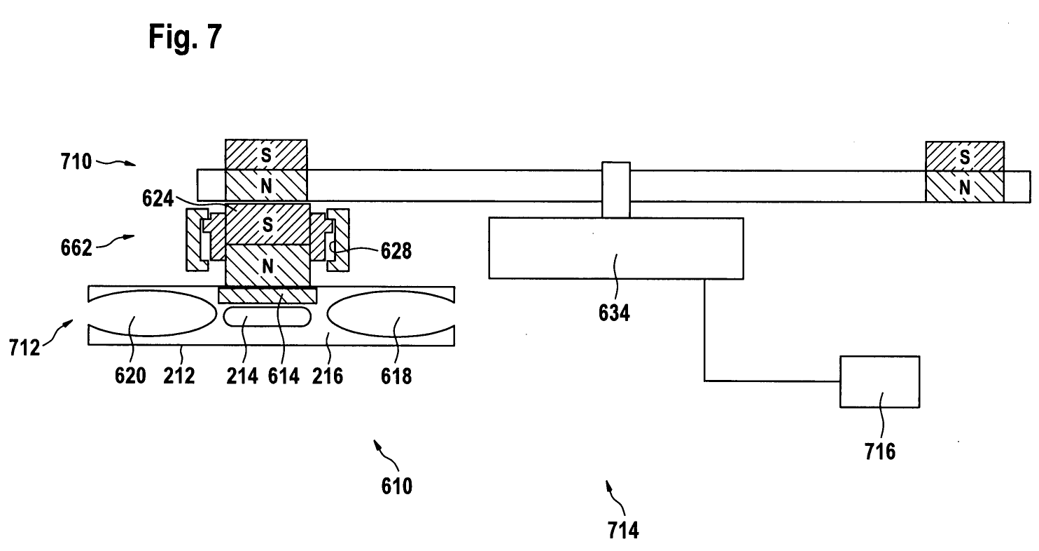

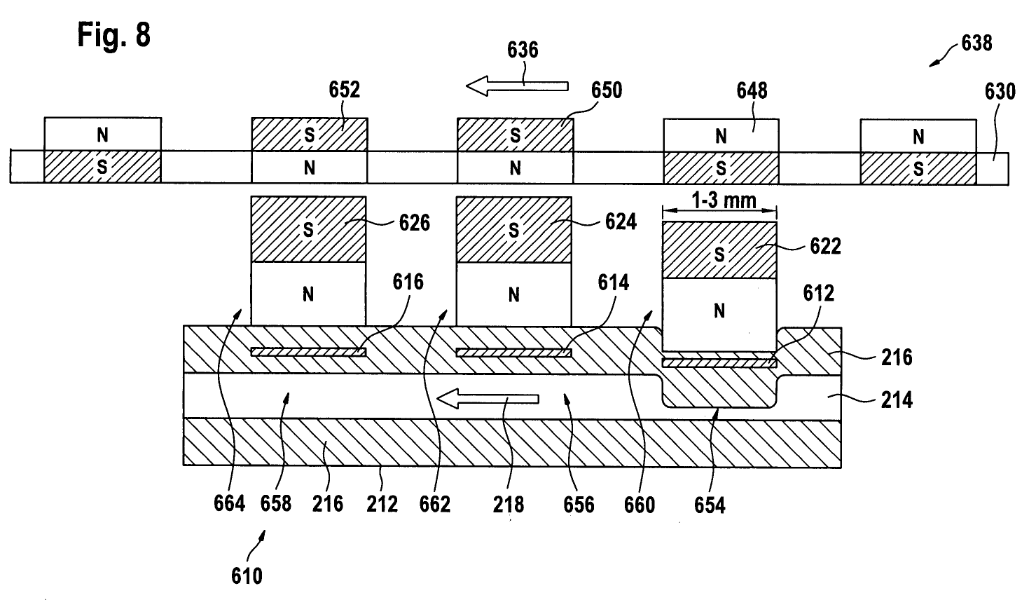

FIGS. 6 to 8 illustrate a third exemplary embodiment of a preferred micropump 610 with three magnetic actuators 660, 662 and 664 for the pumping of fluidic media. In this case, FIG. 6 illustrates a top view of the micropump 610, FIG. 7 a sectional illustration perpendicularly to the direction of flow 218 and FIG. 8 a sectional illustration parallel to the direction of flow 218.

The micropump 610 again has a casing 212 and a flow duct 214 with a wall body 216 consisting of silicone. Above the flow duct 214, three round soft-iron wafers 612, 614 and 616 are sealed into the wall body 216. These soft-iron wafers are in this case spaced apart equidistantly in the direction of flow 218.

In addition to the flow duct, two further relief ducts 618 and 620, running parallel to the flow duct 214, are introduced. These relief ducts make it easier for the wall body 216 to be pressed together. For the sake of simplification, these relief ducts are not illustrated in FIG. 8. Three bar-shaped permanent magnets 622, 624 and 626 are arranged above the soft-iron wafers 612, 614 and 616 outside the casing 212, the magnetic orientation being identical in all the bar magnets. The bar magnets 622, 624 and 626 in each case have a diameter of approximately 1-3 mm. In this exemplary embodiment, the magnetic south pole is towards the top in all the bar magnets. The bar magnets are mounted in a mechanical guide 628 in such a way that they can move to and fro in each case between an upper and a lower position. The mechanical guide 628 is illustrated only in FIG. 7 for the sake of simplification. As shown in FIG. 8, in an illustrated photograph of the exemplary embodiment, the bar magnets 624 and 626 are in an upper position, but the bar magnet 622 is in a lower position.

Above the bar magnets is arranged a rotor disc 630 which can be set in rotation (or at least in a rotational movement over a predetermined angular sector) by an electric motor 634 via a shaft 632. In the example illustrated, the electric motor 634 rotates in such a way that the direction of rotation 636 of the rotor disc 630 is clockwise in FIG. 6. For the sake of simplification, the shaft 632 and the electric motor 634 are not illustrated in FIG. 8. The electric motor may additionally also be provided with a gear (not illustrated). Both controllable direct-current and alternating-current motors and stepping motors may be used. Instead of an electric motor, other actuators may also be employed which set the rotor discs 630 in a rotational movement, for example piezoelectric drives.

24 bar-shaped permanent magnets 638 are fastened on the rotor disc 630 along the circumference of the latter. The bar-shaped permanent magnets 638 are in this case arranged in the same magnet preferred direction in such a way that pairs with identical magnetic polarization always come to lie next to one another, followed in each case by a pair with opposite magnetic polarization. Thus, in the example illustrated in FIG. 6, the magnets 640 and 642 are arranged with the magnetic south pole directed upwards, whereas the adjacent pair of magnets 644 and 646 is arranged with the magnetic south pole directed downwards. Overall, 24 magnets 638 of this type are arranged along the circumference.

Alternatively, in the example illustrated, the bar-shaped permanent magnets 638 could also be arranged in such a way that in each case two permanent magnets 638, similar to the magnets 650, 652, would be adjacent with an upper south pole, followed by only one permanent magnet 638 with a lower south pole (similar to 648). The advantage of this is that then, in any position of the rotor disc 630, that is to say even in “intermediate positions”, in which no permanent magnet is assigned unequivocally to one of the pumping magnets 622, 624 or 626, there is always at least one of the pumping magnets 622, 624 and 626 which is pressed onto the wall body 216 and closes a pinch region.

As illustrated in FIG. 8, the radius of the rotor disc 630 and the spacing of the bar-shaped permanent magnet 638 are selected exactly such that they correspond to the spacing of the bar-shaped permanent magnet 622, 624 and 626. Thus, in the photograph illustrated in FIG. 8, the bar-shaped permanent magnet 648 comes to lie above the bar-shaped permanent magnet 622, the magnet 650 above the magnet 624 and the magnet 652 above the magnet 626. Although there is not an absolute position match on account of the arrangement of the bar-shaped permanent magnets 638 on a circular path, there is nevertheless a substantial match.

This uniform spacing of the magnets 638 on the rotor disc 630 has a disadvantage however, that at least one of the pinch regions 654, 656, 658 is not closed reliably at each time point. Thus, for example in FIG. 8, when the rotor disc 630 is in an angular position in which in each case a magnet 638 with a lower north pole (for example, 648) is located exactly “between” the magnets 622 and 624 and also between 624 and 626, the middle pinch region 656 is unequivocally opened, since the pumping magnet 624 is pulled upwards. However, the two outer pumping magnets 622 and 626 are located exactly between an attracting and a repelling magnet 638 and are therefore in an “undefined” position. In this position, therefore, none of the pinch regions 654, 656, 658 is reliably closed.

This problem can be overcome, for example, by means of a non-equidistant arrangement of the pumping magnets 622, 624 and 626. If, for example in the above-described “intermediate position” of the rotor disc 630, the pumping magnet 622 is displaced to the right in FIG. 8 by an amount of 0.1 to 0.4 times the distance between the magnets 630, this ensures that, in the described angular position of the rotor disc 630, this magnet 622 is pressed down and thus closes the pinch region 654.

The functioning of the micropump of this third exemplary embodiment can be explained, in particular, with reference to FIG. 8. The moveably mounted bar-shaped permanent magnets 622, 624 and 626 are in each case attracted or repelled by the bar-shaped permanent magnets 648, 650 and 652 lying above them. In the event of repulsion, the respective bar-shaped permanent magnet is pressed into a lower position, in which case it acts mechanically on the wall body 216. The flow duct 214 is at the same time narrowed, and the narrowing may be to an extent such that the flow duct is closed completely in this region. Three possible pinch regions 654, 656 and 658 are thereby formed according to the position of the permanent magnets 622, 624 and 626. In the event of a corresponding narrowing of the cross section of the flow duct 214, fluid which is located in the said flow duct is displaced out of the respective pinch region.

By contrast, when the bar-shaped permanent magnets 622, 624 or 626 are moved into an upper position, the wall body 216 is, on the one hand, relieved of the mechanical pressure. Moreover, the respective soft-iron wafers 612, 614 or 616 are attracted by the respective bar-shaped permanent magnets 622, 624 or 626, thus leading to an additional mechanical widening of the flow duct 214 in the respective associated pinch region 654, 656 or 658. The mechanical attraction therefore has the effect that the return of the wall body 216 does not take place solely on account of elastic return forces of the wall body 216, but is additionally “magnetically assisted”.

Alternatively, as described above, instead of the soft-iron wafers 612, 614, 616, iron powder or carbonyl iron powder could also be incorporated locally into the wall body 216.

In the event of a corresponding rotational movement of the rotor disc 630, the bar-shaped permanent magnets 622, 624 and 626 move cyclically up and down. The respective pinch regions 654, 656 and 658 are thereby cyclically pinched together and widened. The arrangement of the bar-shaped permanent magnets 638 on the rotor discs 630 ensures that at least one pinch region is narrowed at any moment.

The operative principle of the micropump 610 is therefore identical to the operative principle in the two preceding exemplary embodiments. A peristaltic pumping movement occurs, fluid being conveyed from right to left in FIG. 8. A reversal of the direction of rotation correspondingly causes a reversal of the direction of flow. The micropump illustrated can thereby be changed over from delivery operation to suction operation, or vice versa, in a simple way. The maximum volumetric flow rate is governed, in particular, by the overall size and the configuration of the actuators and also by the rotational frequency of the rotor disc 630 and may attain the ml/min range. The micropump 610 is particularly suitable for volumetric flow rates in the range of 0 to 1000 nl/min, particularly when pulsating flows are permitted.

Since force transmission takes place by pressure during the closing of the actuator and by magnetic forces during opening, there is no need for a fixed mechanical connection between the pump body (in particular, the casing 212 or the wall body 216) and the pumping magnets 622, 624 and 626. The pump can consequently be divided, in turn, into two parts (see FIG. 7): the drive subassembly 710 and the fluidic subassembly 712. The drive subassembly 710 comprises the rotor 630 with the drive 632, 634 and with the bar-shaped permanent magnets 638, and also the three pumping magnets 622, 624 and 626. The fluidic subassembly 712 comprises the casing 212 with wall body 216 and flow duct 214 and the soft-iron wafers 612, 614 and 616. Whilst the drive subassembly 710 can be designed to be reusable, the fluidic subassembly 712 can also be designed, in particular, as an inexpensive disposable part, so that complicated cleaning after use may be dispensed with.

The third exemplary embodiment illustrated can be extended to a larger number of actuators in a simple way, similarly to the two preceding exemplary embodiments. Thus, for example, it would be expedient, in the case of four magnetic actuators, to arrange the bar-shaped permanent magnets 638 on the rotor disc 630 in groups of two having an identical magnetic orientation. This would ensure that the flow duct 214 is narrowed in its cross section in each case in additionally at least one pinch region. A higher pumping pressure can thereby be achieved.

The micropump described, in one of its embodiments, in particular a micropump which has been produced in one of its embodiments by the method according to the invention, can be employed advantageously in numerous fields of medical technology, process engineering and chemistry. Thus, the micropump can be employed in diagnostics, especially in medical diagnostics, or for medical metering systems, in particular for active-substance and analgesic metering systems, for example for the metering of insulin.

Furthermore, the micropump may also be employed in chemical microreactor technology, and for metering systems for reagents or auxiliaries, for example lubricants.

The micropump described affords a series of advantages, as compared with conventional pumps. Thus, the micropump allows, in particular, suction and delivery operation. In this case, the pumping capacity, in particular the volumetric throughput, can be fully controlled. The micropump is also suitable, in particular, for a low volumetric throughput, for example in the range of 0 to 10 000 nl/min, sometimes even down to a volumetric throughput of 0 to 100 nl/min. The pumping direction is in this case reversible at any time. The volumetric flow rate remains virtually constant over long periods of time, even without any regulating intervention by the user, and only a slight temperature and pressure dependence of the volumetric flow rate is to be noted.

Furthermore, the micropump has an extremely small overall size and also a high further miniaturization potential. Thus, the micropump described is, for example, considerably smaller than the pump which is described in DE 100 29 453 C2 and which is suitable for a similarly low volumetric throughput. Moreover, the micropump described can be integrated into fluidic microstructures in a simple way. In particular, the low production costs are also advantageous, as compared with known diaphragm-type peristaltic and diffuser pumps.

The substantial independence of the pumping speed from the external pressure and from the external temperature is advantageous, as compared with the pumps operating with gas as the conveying medium. The micropump described, in one of its embodiments, is suitable equally for gases and for liquids, even gas bubbles being acceptable.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may very from a stated reference without resulting in a change in the basic function of the subject matter at issue.

Having described the invention in detail and by reference to specific embodiments thereof, it will be apparent that modification and variations are possible without departing from the scope of the invention defined in the appended claims. More specifically, although some aspects of the present invention are identified herein as preferred or particularly advantageous, it is contemplated that the present invention is not necessarily limed to these preferred aspects of the invention.

What is claimed is:

1. A micropump for pumping of a fluidic media, the micropump comprising:
at least one flow duct having at least one wall body;
at least one pinch region in the at least one flow duct; and
at least one actuator, wherein the at least one actuator is capable of exerting force in the at least one pinch region such that a cross-sectional area of the at least one flow duct being capable of being varied in the at least one pinch regions by the force exerted by the at least one actuator.

2. The micropump according to claim 1, further comprising at least one casing, wherein the at least one casing completely or partially surrounds the at least one flow duct.

3. The micropump according to claim 1, wherein the at least one flow duct has a round or elliptical or lenticular cross-sectional area, such that a radius or lengths of a semi-axes of the cross-sectional area is in a range of between 10 micrometers and 1000 micrometers.

4. The micropump according to claim 1, wherein the number of at least one actuators corresponds to the number of at least one pinch region.

5. The micropump according to claim 1, wherein when the force is exerted on the at least one flow duct in at least one pinch region, a fluid located in the at least one flow duct is displaced completely or partially out of the at least one pinch region.

6. The micropump according to claim 1, wherein the at least one wall body comprises completely or partially an elastomer material.

7. The micropump according to claim 1, wherein the at least one wall body comprises completely or partially a silicone.

8. The micropump according to claim 7, wherein the silicone is a cold-curable dimethylsiloxane.

9. The micropump according to claim 1, wherein the at least one pinch region is arranged along a longitudinal axis of the at least one flow duct.

10. The micropump according to claim 1, further comprising at least three pinch regions and at least three actuators.

11. The micropump according to claim 10, wherein the force exerted by the at least three actuator in the at least three pinch regions takes place periodically at an actuator frequency which is identical for all the actuators.

12. The micropump according to claim 1, wherein the at least one flow duct has at least two connection regions, wherein the at least two connection regions are for connecting the at least one flow duct to a supply system and a discharge system for the fluidic media.

13. The micropump according to claim 1, having a volumetric flow rate of between 0 to 1000 nl/min.

14. The micropump according to claim 1, wherein at least one actuator is a bimetal actuator.

15. The micropump according to claim 14, wherein the shape of the bimetal actuator can be varied as a result of the application of an electric voltage and/or the conduction of an electrical current such that the magnitude of the force exerted on the at least one flow duct is set by means of the intensity of the electrical voltage and/or of the electrical current.

16. The micropump according to claim 14, wherein the bimetal actuator has a tongue shape.

17. The micropump according to claim 1, wherein at least one actuator is a thermal actuator.

18. The micropump according to claim 17, wherein the thermal actuator comprises at least one heat source and a homogeneous expansion region of the at least one wall body, wherein the at least one heat source is capable of locally raising the temperature of the homogeneous expansion region, such that density of the homogeneous expansion region of the at least one wall body being locally reduced that results in narrowing the cross-sectional area of the at least one flow duct in the at least one pinch region.

19. The micropump according to claim 18, wherein the at least one heat source is an electrically heatable resistor, wherein a temperature of the electrically heatable resistor is capable of being set as a result of an application of an electrical voltage and/or the conduction of an electrical current such that the degree of narrowing of the cross-sectional area of the at least one flow duct is set by the intensity of the electrical voltage and/or of the electrical current.

20. The micropump according to claim 17, wherein the thermal actuator has an SMD resistor.

21. The micropump according to claim 17, having a double-walled theremally insulating casing such that the thermally insulating casing completely or partially surrounds the at least one pinch region and the thermal actuator.

22. The micropump according to claim 17, wherein the at least one wall body has a material which has a linear coefficient of thermal expansion of between 0.01 and 0.09%/K.

23. The micropump according to claim 1, wherein the at least one actuator is a magnetic actuator.

24. The micropump according to claim 23, wherein the magnetic actuator comprises:
at least one force transmission element integrated into the at least one wall body such that at least one force transmission element being configured in such a way that a force can be exerted on the force transmission element as a result of the action of a magnetic field;
at least one magnetic-field generation element, wherein the at least one magnetic-field generation element generating a magnetic field of variable intensity; and
wherein the magnetic field being capable of acting on the at least one force transmission element.

25. The micropump according to claim 24, wherein the force transmission element has a ferromagnetic material.

26. The micropump according to claim 24, wherein the force transmission element has a ferromagnetic powder, the ferromagnetic powder being embedded into the at least one wall body.

27. The micropump according to claim 24, wherein the magnetic-field generation element further comprises:
at least one pumping magnet, wherein the at least one pumping magnet is capable of being variable in its spatial position and/or its orientation;
at least one pumping magnet generating a magnetic field which acts on the at least one force transmission element, wherein the magnetic field generated by the at least one pumping magnet at the location of the at least one force transmission element being dependent on the position and/or orientation of the at least one pumping magnet; and
at least one control magnet, that is capable of setting the spatial position and/or the orientation of the at least one pumping magnet.

28. The micropump according to claim 27, wherein the at least one pumping magnet has at least one permanent magnet.

29. The micropump according to claim 28, wherein all the permanent magnets have a common magnetic orientation.

30. The micropump according to claim 27, wherein the at least one pumping magnet has at least two different positions or orientations, such that the at least one pumping magnet acting mechanically on at least one wall body in such a way that, in at least one of the at least two positions or orientations of the at least one pumping magnet in at least one pinch region, the at least one flow duct has a cross-sectional area other than in the at least one other position or orientation of the at least one pumping magnet.

31. The micropump according to claim 27, wherein the at least one control magnet comprises:
at least one spatially moveable device, and
at least three magnetic elements connected to the spatially moveable device.

32. The micropump according to claim 31, wherein the spatially moveable device has a rotor disc such that the magnetic elements are arranged on the rotor disc along a circular path.

33. The micropump according to claim 30, wherein the at least three magnetic elements have a common preferred axis and are divided into groups with identical magnetic polarization such that the number of magnetic elements in each group does not overshoot the number of pumping magnets, reduced by one.

34. A micropump for pumping of a fluidic media, the micropump comprising:
a flow duct having a wall body;
a pinch region in the flow duct;
an actuator, wherein the actuator is capable of exerting force in the pinch region such that a cross-sectional area of the flow duct being capable of being varied in the pinch regions by the force; and
an electronic control for activating the actuator.

35. The micropump according to claim 34, further comprising a casing, the casing completely or partially surrounds flow duct.

36. The micropump according to claim 34, wherein the flow duct has a round or elliptical or lenticular cross-sectional area, such that a radius or lengths of a semi-axes of the cross-sectional area is in a range of between 10 micrometers and 1000 micrometers.

37. The micropump according to claim 34, wherein the number actuators corresponds to the number of the pinch region.

38. The micropump according to claim 34, wherein when the force is exerted on the flow duct in the pinch region, a fluid located in the flow duct is displaced completely or partially out of the pinch region.

39. The micropump according to claim 34, wherein the wall body comprises completely or partially of an elastomer material.

40. The micropump according to claim 34, wherein the wall body comprises completely or partially a silicone.

41. The micropump according to claim 40, wherein the silicone is a cold-curable dimethylsiloxane.

42. The micropump according to claim 34, wherein the pinch region is arranged along a longitudinal axis of the flow duct.

43. The micropump according to claim 34, further comprising at least three pinch regions and at least three actuators.

44. The micropump according to claim 34, having a volumetric flow rate of between 0 to 1000 nl/min.

45. The micropump according to claim 34, wherein the actuator is a bimetal actuator.

46. The micropump according to claim 45, wherein the bimetal actuator has a tongue shape.

47. The micropump according to claim 34, wherein the actuator is a thermal actuator.

48. The micropump according to claim 34, wherein the wall body has a material which has a linear coefficient of thermal expansion of between 0.01 and 0.09%/K.

49. The micropump according to claim 1, wherein the actuator is a magnetic actuator.

50. A micropump for pumping of a fluidic media, the micropump comprising:
a flow duct having a wall body;
more than one pinch region in the flow duct; and
more than one actuator, wherein the actuator is capable of exerting force in the pinch region such that a cross-sectional area of the flow duct being capable of being varied in the pinch regions by the force exerted by the actuator.

51. The micropump according to claim 50, wherein:
a number of N pinch regions are distributed along the longitudinal axis of the flow duct;
a flow path of length Xᵢ₋ⱼ occurring between the i'th and the j'th pinch region, with the relation i<j, and i, jε{I, . . . , N}, to apply, and i and j are to be integers;
the phase angle φᵢ₋ⱼ of the phase shift of the force action between the i'th and the j'th pinch region being proportional to the length Xᵢ₋ⱼ with a proportionality factor kᵢ₋ⱼ, and
the proportionality factor kᵢ₋ⱼ being identical for all i, jε{I, . . . , N}.

52. The micropump according to claim 50, wherein at each time point, the flow duct is closed in at least one pinch region.

53. The micropump according to claim 50, wherein the pinch regions are distributed equidistantly along the longitudinal axis, and the phase angle of the phase shift of the force action between two adjacent pinch regions amounts to 360° divided by the number of pinch regions.

54. The micropump according to claim 50, wherein the number of actuators correspond to the number of the pinch region.

55. A method for production of a micropump for the pumping of fluidic media, the method comprising:
producing a casing using the a microinjection-moulding method;
introducing at least one actuator into the casing;
introducing at one flow-duct master moulding into the casing;
filling the casing with an elastic material; and
removing the at least one flow-duct master moulding from the casing.

56. The method according to claim 55, further comprising the step of coating at least one surface of the at least one casing and/or of the at least one actuator with an adhesion promoter layer.

57. The method according to claim 55, further comprising the step of coating at least one surface of the at least one flow-duct master moulding with an anti-adhesion layer.

58. The method according to claim 55, wherein he elastic material is silicone.

59. The method according to claim 58, wherein the silicone is cold-curable dimethylsiloxane.

60. The method according to claim 55, wherein the actuators introduced into the casing is a selected from a group consisting of a bimetal actuator, a thermal actuator or a magnetic actuator.
